(19) United States
(12) Patent Application Publication
(10) Pub. No.: US 2026/0146983 A1
(43) Pub. Date: May 28, 2026

(54) QUANTITATIVE ANALYSIS OF BIOMASS SAMPLES

(71) Applicant: Soliton, LLC, Sioux Falls, SD (US)

(72) Inventor: Tessanna Bauer Schmitz, Chancellor, SD (US)

(21) Appl. No.: 19/124,125

(22) PCT Filed: Oct. 24, 2023
(86) PCT No.: PCT/US2023/035825

§ 371 Date: Apr. 24, 2025

Related U.S. Application Data
(60) Provisional application No. 63/419,196, filed on Oct. 25, 2022.

(51) Int. Cl. G01N 33/00; G01N 1/40; G01N 1/44

(52) U.S. Cl. CPC G01N 33/0098; G01N 1/4044; G01N 1/44

(57) ABSTRACT

A method for quantifying a cellulosic component in a sample includes preparing a first suspension by mixing an amount of sample with first aqueous acid at a concentration of 40 wt-% or greater; incubating the first suspension in a first incubation stage at a first incubation temperature of less than 40° C.; adding water or aqueous sulfuric acid to the incubated first suspension to form a second suspension having a concentration 40 wt-% or less of sulfuric acid based on a total weight of the second suspension; incubating the second suspension in a second incubation stage at a second incubation temperature of 50° C. or greater for an incubation period, wherein the sample is not subjected to hydrolysis at temperatures above 98° C.; separating a supernatant from the incubated second suspension; analyzing the supernatant for total content of xylose, mannose, galactose, and arabinose; and calculating, based on the analysis, the amount of the hemicellulose.

### CROSS-REFERENCE TO RELATED APPLICATIONS

This application claims the benefit of U.S. Provisional Application No. 63/419,196 filed Oct. 25, 2022, the disclosures of which are incorporated by reference herein in their entireties.

### FIELD

The present disclosure relates to methods for compositional analysis of cellulosic materials and feedstocks. The present disclosure further relates to methods for compositional analysis of corn-based samples and other similar materials.

### BACKGROUND

Lignocellulosic biomass is a complex material that is made up of three main structural polymers: cellulose, hemicellulose, and lignin. Cellulose is a linear polysaccharide containing linked β-1,4-glucose units. The glucose units are linked to one another by hydrogen bonds and can form a crystalline or amorphous structure. Hemicellulose is a heteropolymer containing xylan, mannan, and glucans in different amounts depending on the source. Hemicelluloses are amorphous and include branched chains that are shorter than the chains in cellulose (about 500 to 3000 sugar units compared to 7000 to 15000 sugar units in cellulose). Xylans are primarily made up of xylose residues but may also be substituted with other sugars, such as glucose and arabinose. Mannans include two types, galactomannans made up of mannopyranose, and glucomannans made up of mannopyranose and glucopyranose. Glucans include mixed linkage β-glucans and xyloglucans, made up of glucose residues or glucose and xylose residues, respectively.

Cellulosic feedstocks can be broken down to their components and used to manufacture various chemicals, such as fuel ethanol. The term “conversion” is often used in the industry to refer to a material's conversion to ethanol. Thus, “cellulosic conversion” typically refers to cellulose or cellulosic material converted to ethanol, and “starch conversion” refers to the conversion of starch to ethanol. In order for manufacturers of cellulosic ethanol to access the financial incentives created by the federal and state programs, the Environmental Protection Agency (“EPA”) Renewable Fuel Standard (“RFS”) and California Air Recourses Board (CARB) Low Carbon Fuels Standard (LCFS) requires analytical verification that the carbohydrates converted to ethanol originate from non-starch (i.e., cellulosic) material.

### SUMMARY

A method for quantifying hemicellulose in a sample includes preparing a first suspension by mixing an amount of sample with aqueous acid comprising zinc chloride at a concentration of 60 wt-% to 80 wt-% or sulfuric acid at a concentration of 40 wt-% to 75 wt-%, based on a total weight of the first suspension; incubating the first suspension in a first incubation stage at a first incubation temperature of less than 40° C. for 10 min to 60 min; adding water or aqueous sulfuric acid to the incubated first suspension to form a second suspension having a concentration of 5 wt-% to 40 wt-% of sulfuric acid based on a total weight of the second suspension; incubating the second suspension in a second incubation stage at a second incubation temperature of 50° C. or greater for an incubation period of 120 min to 500 min, wherein the sample is not subjected to hydrolysis at temperatures above 98° C.; separating a supernatant from the incubated second suspension; analyzing the supernatant for total content of xylose, mannose, galactose, and arabinose; and calculating, based on the analysis, the amount of the hemicellulose.

The sample may be corn-based. The sample may include corn kernel fiber. The sample may include a starting material, an intermediate product, or a final product from a corn-to-ethanol production process.

The amount of sample mixed with an amount of the aqueous sulfuric acid may be from 8 to 12 parts of aqueous sulfuric acid to every 1 part sample. The sample may not be subjected to hydrolysis at temperatures above 90° C. The sample may be only subjected to temperatures of 90° C. or lower during the method. The supernatant may be substantially free of furfural and hydroxymethylfurfural.

The method may include calculating hemicellulose as:

$$\% dw Total Hemicellulose = ( \frac{( \frac{X - Y + S - T}{100} ) * ( A + R )}{A} ) / ( 1.136 * ( \frac{D}{100} ) ) * 100 ,$$

where:
R is a total weight of liquid added to the sample;
A is mass of the sample;
D is wt-% total solids content of the sample;
S is the arabinose content of the sample in wt-%;
T is an arabinose content of a blank in wt-%;
X is a combined xylose, galactose, and mannose content of the sample in wt-%; and
Y is a combined xylose, galactose, and mannose content of the blank in wt-%.

An analysis result including a hemicellulose concentration of a sample may be obtained by: preparing a first suspension by mixing an amount of sample with aqueous acid comprising zinc chloride at a concentration of 60 wt-% to 80 wt-% or sulfuric acid at a concentration of 40 wt-% to 75 wt-%, based on a total weight of the first suspension; incubating the first suspension in a first incubation stage at a first incubation temperature of less than 40° C. for 10 min to 60 min; adding water or aqueous sulfuric acid to the incubated first suspension to form a second suspension having a concentration of 5 wt-% to 40 wt-% of sulfuric acid based on a total weight of the second suspension; incubating the second suspension in a second incubation stage at a second incubation temperature of 50° C. or greater for an incubation period of 120 min to 500 min, wherein the sample is not subjected to hydrolysis at temperatures above 98° C.; separating a supernatant from the incubated second suspension; analyzing the supernatant for total content of xylose, mannose, galactose, and arabinose; and calculating, based on the analysis, the amount of the hemicellulose.

### BRIEF DESCRIPTION OF FIGURES

FIG. 1 is a graphical representation of the fermentation kinetics of both C5 and C6 sugars derived from cellulose and hemicellulose as described in EXAMPLE 2.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

The term “cellulosic content” is used here to refer to the combination of cellulose, hemicellulose, and lignin.

The term “cellulosic components” is used here to refer to cellulose and hemicellulose. “A cellulosic component” is intended to mean either cellulose or hemicellulose, individually.

The term “cellulosic material” is used here to refer to materials that contain cellulosic components. Examples of cellulosic materials include various grains, such as corn, wheat, sorghum, barley, and the like, as well as various types of biomass, such as wood, stover, straw, hay, grass, and by-products and end products from various processes that use grains or biomass, such as fermentation, digestion, and other conversion processes.

The term “fiber” is commonly used in the industry to refer to both the fiber fraction of grains, such as corn, as well as an analytical parameter. Here, the fiber fraction of corn is referred to as “corn kernel fiber” and the analytical parameter is referred to as “fiber” The term “substantially” as used here has the same meaning as “significantly,” and can be understood to modify the term that follows by at least about 90%, at least about 95%, or at least about 98%.

The term “substantially free” is used here to mean that the compound in question is present in the composition at a level of 1 wt-% or lower, including 0 wt-%.

The term “not substantially” as used here has the same meaning as “not significantly,” and can be understood to have the inverse meaning of “substantially,” i.e., modifying the term that follows by not more than 25%, not more than 10%, not more than 5%, or not more than 2%.

The term “about” is used here in conjunction with numeric values to include normal variations in measurements as expected by persons skilled in the art, and is understood to have the same meaning as “approximately” and to cover a typical margin of error, such as ±5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

### DETAILED DESCRIPTION

The present disclosure relates to methods for compositional analysis of cellulosic materials and feedstocks. In particular, the present disclosure relates to methods for analysis of hemicellulose and cellulose content in cellulosic materials and feedstocks. The present disclosure further relates to methods for compositional analysis of corn kernel fiber and other similar materials.

Quantitation of cellulose, hemicellulose, fiber, and lignin in lignocellulosic materials is desired, for example, because in order to claim government benefits available to producers of renewable cellulosic fuels (e.g., ethanol), the producers must differentiate between starch-based fuels and fuels derived from cellulosic components. Many producers use an in-situ cellulosic conversion, where cellulosic sugars become mixed with starch-based sugars prior to conversion to ethanol. Other producers use fractionated corn kernel fiber or post-conversion materials (e.g., after conversion of starch to ethanol) as their feedstock in the cellulosic conversion process. Post-conversion materials from corn-to-ethanol fermentation typically contain corn kernel fiber, germ, and other residual materials. Depending on the efficiency of the conversion process, the post-conversion material may also contain some starch. The starch-based fuels and fuels derived from cellulosic components can be differentiated by analyzing the feedstock used in the fuel production and reporting the relative amounts of starch and cellulosic components converted to fuel.

The EPA identifies ethanol produced from cellulosic materials by a renewable identification number (“RIN”) D3. Thus, the relative amount of ethanol produced from cellulosic content versus starch is sometimes referred to as “% D3 RIN.” The California Air Resources Board (“CARB”) has also implemented a Low Carbon Fuel Standard (“LCFS”) that requires fuels supplied for use in California to meet certain carbon intensity standards. Cellulosic ethanol qualifies as a Low Carbon Intensity (“CI”) Fuel under the CARB LCFS.

Quantitation of cellulose, hemicellulose, fiber, and lignin in lignocellulosic materials is challenging due to the varied chemical structures within the materials and the compounds of interest, the lack of analytical methods to directly measure the compounds of interest, and the difficulty in breaking down the compounds into measurable components without causing degradation or unintended chemical reactions.

Methods for determining carbohydrate composition in lignocellulosic materials typically include various steps that attempt to quantitatively saccharify the various compounds within the material to quantifiable components, such as simple sugars. The National Renewable Energy Laboratory (“NREL”) has developed various test methods over the years (including versions released 1996, 2006, and 2021) that are widely used in the renewable fuels industry. Typical compositional methods for biomass analysis involve a primary hydrolysis in strong (e.g., 72 wt-%) sulfuric acid at a low temperature (ambient to 30° C.) to convert polysaccharides into oligosaccharides, and a secondary hydrolysis in weak (e.g., 3 wt-% to 4 wt-%) sulfuric acid at a high temperature (e.g., boiling to 125° C.) and optionally at high pressure to convert the oligosaccharides to monosaccharides. The handling of samples in concentrated sulfuric acid and of boiling sulfuric acid under pressure cause the need for additional laboratory safety measures and specialized equipment.

The analytical methods currently used to quantify cellulose and hemicellulose are complex and difficult to perform and may require specialized equipment.

Some researchers have suggested adding a parallel control to account for degradation of sugars during analysis. See, e.g., Moxley and Zhang, More Accurate Determination of Acid-Labile Carbohydrates in Lignocellulose by Modified Quantitative Saccharification, 21 Energy & Fuels 3684 (2007). Moxley and Zhang also point out that different sugars degrade at different rates in the acid hydrolysis conditions.

The existing methods and added improvements still result in complicated analytical methods. The commonly used acid hydrolysis with concentrated sulfuric acid presents safety concerns and causes a need for additional precautions at laboratories. The acid hydrolysis performed with weaker acid but at high temperature and pressure requires specialized equipment, adding to the cost of analysis.

It would be desirable to provide an analytical method that can be performed without specialized equipment. It would further be desirable to provide an analytical method that does not result in sugar degradation during the method. It would further be desirable to provide an analytical method that can provide the hemicellulose content of a sample.

According to an embodiment, a method for compositional analysis of cellulosic materials is provided. The method includes a dual stage hydrolysis step that can be performed at ambient pressure and below the boiling point of water. The method may be performed without specialized equipment. The method may be used to quantify hemicellulose in cellulosic materials, particularly in corn-based cellulosic materials, such as raw ground corn and starting materials and residual materials from corn-to-ethanol processes. The method may be used to quantify hemicellulose and to calculate the amount of cellulose by subtracting the hemicellulose and lignin from the total cellulosic content. It should be noted that in corn (e.g., #2 Yellow corn), the amount of lignin is zero or substantially zero. That is, the total cellulosic content of corn is substantially the same as the cellulosic components content. The method does not cause any significant degradation of sugars during the method.

The methods of the present disclosure are particularly useful for quantifying hemicellulose in corn-based cellulosic materials. The cellulosic components of corn are primarily present in corn kernel fiber. The hemicellulose in corn is mostly made up of xylose, mannose, galactose, and arabinose residues and does not contain significant amounts of glucose residue. The methods of the present disclosure are particularly useful for quantifying hemicellulose in corn and corn-based products.

According to an embodiment, the method utilizes a dual stage acid hydrolysis process and a low temperature (below the boiling point of water) at ambient pressure in order to break down the cellulosic material while avoiding degradation of the sugars. Ambient pressure is intended to mean normal atmospheric pressure, although some minor pressure build-up may occur in capped test tubes when heated to the hydrolysis temperature. The method does not include, however, the use of a pressurized enclosure, such as a closed autoclave heated to 100° C. or higher. The method includes quantification of hemicellulose or both cellulose and hemicellulose present in the initial sample. In some embodiments, the method includes calculation of the amount of cellulose.

According to an embodiment, the method may be used to analyze samples of cellulosic material. The sample may be a grain-based sample. Some non-limiting examples of grains include corn, sorghum, wheat, barley, and the like. While any types of grain-based samples may be analyzed using the method, the method may be particularly useful for analyzing grain-based samples in conjunction with renewable fuel production. In some embodiments, the sample is corn, a fraction of corn, or a residual of corn. In some embodiments, the sample contains corn kernel fiber. The sample may be primarily corn kernel fiber. The sample may be a starting material, an intermediate product, or a final product from an ethanol production process, such as a corn-to-ethanol production process (e.g., a dry grind ethanol process) or a sorghum-to-ethanol production process. Examples of a starting material from a corn-to-ethanol production process is corn, corn flour, a corn fraction, or the like, mash, or a slurry substrate. In some embodiments, the sample is or contains raw ground corn (e.g., corn flour). Raw ground corn is understood to mean corn that has been ground but not cooked or treated, e.g., enzymatically. An example of an intermediate product from a corn-to-ethanol production process is wet cake, which is a post-fermentation mixture of remaining solids and liquid. An example of a final product from a corn-to-ethanol production process is dried distiller's grains (optionally with solubles), DDG(S), which is a dried post-fermentation mixture of remaining solids, optionally mixed with soluble solids Similar corresponding starting materials, intermediate products, and final products may be analyzed from a sorghum-to-ethanol process. The sample may also be any number of animal feed or human food products. In particular, the sample may be a grain-based feed or food product.

The samples may be prepared for analysis by drying, grinding, or both drying and grinding. Samples that have a moisture content of 10 wt-% or greater may be dried, for example, in an oven or by another suitable dehydration process without increasing the sample temperature above 50° C. According to an embodiment, the sample has a moisture content of less than 10 wt-% prior to beginning the compositional analysis. Alternatively, the moisture content of the sample is as-is, even if the moisture content is higher than 10 wt-%. In such cases, the amount and concentration of acid is adjusted so that a desired acid concentration in the hydrolysis solution is reached.

Samples that cannot pass (at least 95 wt-%) through a 0.5 mm screen are ground by any suitable method such that at least 95 wt-% of the sample passes through a 0.5 mm screen, with the remainder passing through a 1 mm screen. According to an embodiment, at least 95 wt-% of the sample passes through a 0.5 mm screen, with the remainder passing through a 1 mm screen prior to beginning the compositional analysis.

The compositional analysis may be performed on any suitable sample size. For practical reasons, a sample size between 0.5 g and 5 g (e.g., 1.0 g±0.10 g) may be used. However, the method is not particularly limited by the sample size, and the amounts of other reagents may be adjusted according to the sample size used.

The method of the present disclosure includes a dual stage acid hydrolysis. The two stages include a first incubation stage and a second incubation stage. During both acid hydrolysis stages, a sample is incubated in a hydrolysis solution containing an acid. The hydrolysis solution during both stages is an aqueous solution. The sample and the hydrolysis solution may be mixed to form a suspension. According to an embodiment, a first suspension is prepared by mixing an amount of sample with a first aqueous acid. The first aqueous acid may be present in the first suspension at a concentration of 40 wt-% or greater. The first aqueous acid may be zinc chloride or sulfuric acid. In embodiments, where the first aqueous acid is zinc chloride, the acid may be present in the first suspension at a concentration of 60 wt-% to 80 wt-%. In embodiments, where the first aqueous acid is sulfuric acid, the acid may be present in the first suspension at a concentration of 40 wt-% to 75 wt-%.

The acid concentration is then diluted for the second incubation stage to a concentration of less than 40%. According to an embodiment, a second suspension is prepared by mixing water or aqueous sulfuric acid with the incubated first suspension. In embodiments where the first suspension is prepared with zinc chloride, the second suspension may be prepared by mixing aqueous sulfuric acid with the first suspension. In embodiments where the first suspension is prepared with sulfuric acid, the second suspension may be prepared by mixing water or more dilute aqueous sulfuric acid with the first suspension. The preparation of the second suspension dilutes the acid concentration of the first suspension. The second suspension has an acid concentration of 40 wt-% or less. In some embodiments, the second suspension has an acid concentration of 5 wt-% or greater, 8 wt-% or greater, 10 wt-% or greater, 15 wt-% or greater, or 20 wt-% or greater. The second suspension may have an acid concentration of 40 wt-% or less, 30 wt-% or less, 25 wt-% or less, 20 wt-% or less, or 15 wt-% or less. The second suspension may have an acid concentration of 5 wt-% to 40 wt-%, 8 wt-% to 20 wt-%, or about 10 wt-%. The acid concentration of the second suspension may be made up of sulfuric acid or a combination of sulfuric acid and zinc chloride. In some embodiments, the second suspension has a concentration of 5 wt-% to 40 wt-%, 8 wt-% to 20 wt-%, or about 10 wt-% of sulfuric acid.

The amount of aqueous acid (volume per weight) mixed with the sample to prepare the first suspension may be about 10 parts acid to every 1 part sample, or from 8 to 12 parts acid to every 1 part sample. The exact volumetric amount of acid is not particularly consequential as long as it is sufficient to hydrolyze the sample. However, the amount of acid is recorded for calculation of the final results of the analysis.

According to an embodiment, the first suspension is incubated in the first incubation stage at a first incubation temperature for a first incubation time. The first incubation temperature may be 40° C. or less, 35° C. or less, 30° C. or less, or 25° C. or less. The first incubation temperature may be ambient temperature (e.g., about 25° C.). The first incubation temperature may be lower than ambient temperature, such as about 4° C. or greater. In some embodiments where the acid used in the first incubation stage is zinc chloride, the first incubation temperature is ambient temperature, or a temperature ranging from 18° C. to 30° C. or from 20° C. to 25° C. In some embodiments where the acid used in the first incubation stage is sulfuric acid, the first incubation temperature is below ambient temperature, such as 2° C. to 21° C., 2° C. to 18° C., 4° C. to 21° C., 4° C. to 18° C., or 4° C. to 10° C. According to an embodiment, no external heat is applied to the first suspension. The first incubation time may be 10 min or greater, 15 min or greater, 20 min or greater, or 30 min or greater. The first incubation time may be 60 min or less, 45 min or less, or 30 min or less. The first incubation time may range from 10 min to 60 min, or from 15 min to 45 min.

According to an embodiment, the second suspension is incubated in the second incubation stage at a second incubation temperature for a second incubation time. The second incubation temperature may be 50° C. or greater, 60° C. or greater, 70° C. or greater, 75° C. or greater, or 80° C. or greater. The second incubation temperature may be 98° C. or lower, 95° C. or lower, 90° C. or lower, or 85° C. or lower. In some embodiments, the second incubation temperature is from 50° C. to 98° C., 60° C. to 95° C., or from 70° C. to 90° C. The second incubation time may be 120 min or greater, 150 min or greater, 200 min or greater, or 360 min or greater. The second incubation time may be 500 min or less, 420 min or less, or 360 min or less. The second incubation time may range from 120 min to 500 min, or from 240 min to 420 min.

The samples may be mixed during the first and/or second incubation stage to ensure exposure of all the sample particles to acid. For example, the samples may be mixed periodically throughout the incubation period, such as every 30 min or 60 min.

According to an embodiment, the hydrolysis is performed at conditions that do not result in substantial degradation of the sugars. That is, the hydrolysis conditions do not result in substantial formation of degradation products, such as furfural and hydroxymethylfurfural (“HMF”). After incubation of the sample in the hydrolysis solution, the sample forms a composition that contains monomeric sugars but is substantially free of furfural and HMF.

After incubation of the sample in the first incubation stage and second incubation stage, a supernatant may be separated from the suspension. Any suitable separation method may be used. For example, to separate the supernatant from any suspended solids, the sample may be centrifuged and filtered. The supernatant may be separated and analyzed for total content of xylose, mannose, galactose, and arabinose. In some embodiments, the supernatant is analyzed for xylose, mannose, galactose, and arabinose content using, for example, a chromatographic method, such as HPLC. Any suitable HPLC method may be used. The amounts of the sugars obtained by HPLC analysis may be adjusted using a weight gain factor. The weight gain on hydrolysis for pentoses (C5 sugars) is 1.136, and for hexoses (C6 sugars) it is 1.111. In methods where xylose, mannose, and galactose co-elute from the HPLC column, the 1.136×weight gain factor may be used for all of these sugars, although it may result in a slight underestimation of hemicellulose if mannose or galactose are present.

A blank may be run alongside with the samples during analysis to account for any errors during the procedure. A pure xylose standard may also be run alongside the samples to verify good method recovery and confirm the absence of degradation of the monosaccharides.

The amount of hemicellulose in the sample may be calculated using the following formula (I):

$$\% dw Total Hemicellulose = ( \frac{( \frac{X - Y + S - T}{100} ) * ( A + R )}{A} ) / ( 1.136 * ( \frac{D}{100} ) ) * 100 ,$$

where:
A is the mass of the sample,
R is a total weight of liquid added to the sample;
S is the arabinose content of the sample in wt-%;
T is the arabinose content of the blank in wt-%;
X is the combined xylose, galactose, and mannose content of the sample in wt-%;
Y is the combined xylose, galactose, and mannose content of the blank in wt-%;
1.136 is the weight gain on hydrolysis;
D is the wt-% total solids content of prepared sample.

The cellulose content of the sample may be calculated by subtracting the hemicellulose value and lignin from the total cellulosic content. The amount of cellulose in the sample may be calculated using the following formula (II):

$$\% dw Cellulose = \% dw Cellulosic Content - \% dw Hemicellulose - \% dw Lignin .$$

In samples that include no lignin or substantially no lignin (such as corn and many other grains), the amount of cellulose may be calculated using the following formula (II)A:

$$\% dw Cellulose = \% dw Cellulosic Content - \% dw Hemicellulose .$$

According to an embodiment, the method does not include any other hydrolysis steps, such as an enzymatic hydrolysis. For example, the method does not include an enzymatic hydrolysis step before acid hydrolysis. The elimination of the enzymatic hydrolysis step prior to acid hydrolysis, combined with acid hydrolysis at mild conditions, is believed to yield more accurate results for total hemicellulose.

According to an embodiment, the method accurately quantitates the hemicellulose concentration of the sample. The method may be able to quantify cellulose with an accuracy of 90% or greater, 95% or greater, or 98% or greater. The method may be able to quantify hemicellulose with an accuracy of 90% or greater, 95% or greater, or 98% or greater.

To be able to appropriately calculate the hemicellulose content of the sample, the moisture content of the prepare sample is analyzed using any suitable moisture analysis method. Examples of suitable moisture analysis methods include Association of Official Analytical Chemists (“AOAC”) Official Method 930.15 Loss on Drying (Moisture) For Feeds, and AOAC Official Method 935.29 Moisture in Malt. Prior to hydrolysis, the sample may be split and a portion of the sample may be analyzed for starch content using a starch assay. One useful starch assay is the AOAC 996.11 Starch (Total) in Cereal Products.

The result of the method may be an analysis result or analysis report obtained by the method as described herein. The analysis result or analysis report may state the hemicellulose content, cellulose content, or both, of the sample, where the hemicellulose content and calculated cellulose content are obtained by the method as described herein. In some cases, the analysis result or analysis report states the hemicellulose content only. In some cases, the analysis result or analysis report states the cellulose content only. In some cases, the analysis result or analysis report states the hemicellulose content and the cellulose content.

### EMBODIMENTS

The following is a non-limiting list of exemplary embodiments according to the present disclosure.
Embodiment 1 is a method for quantifying hemicellulose in a sample of cellulosic material, the method comprising: preparing a first suspension by mixing an amount of sample with first aqueous acid comprising zinc chloride at a concentration of 60 wt-% to 80 wt-% or sulfuric acid at a concentration of 40 wt-% to 75 wt-%, based on a total weight of the first suspension; incubating the first suspension in a first incubation stage at a first incubation temperature of less than 40° C. for 10 min to 60 min; adding water or aqueous sulfuric acid to the incubated first suspension to form a second suspension having a concentration of 5 wt-% to 40 wt-% of sulfuric acid based on a total weight of the second suspension; incubating the second suspension in a second incubation stage at a second incubation temperature of 50° C. or greater for an incubation period of 120 min to 500 min, wherein the sample is not subjected to hydrolysis at temperatures above 98° C.; separating a supernatant from the incubated second suspension; analyzing the supernatant for total content of xylose, mannose, galactose, and arabinose; and calculating, based on the analysis, the amount of the hemicellulose.
Embodiment 2 is the method of embodiment 1, wherein the sample is corn-based or sorghum-based.
Embodiment 3 is the method of embodiment 1 or 2, wherein the sample comprises raw ground corn.
Embodiment 4 is the method of any one of the preceding embodiments, wherein the sample comprises a starting material, an intermediate product, or a final product from a corn-to-ethanol production process.
Embodiment 5 is the method of any one of the preceding embodiments, wherein the amount of sample is mixed with an amount of the first aqueous acid that is from 8 to 12 parts of aqueous acid to every 1 part sample.
Embodiment 6 is the method of any one of the preceding embodiments, wherein the first incubation temperature is 35° C. or less, 30° C. or less, or 25° C. or less, or wherein the first incubation temperature is ambient temperature (e.g., about 25° C.).
Embodiment 7 is the method of any one of the preceding embodiments, wherein the acid used in the first incubation stage is zinc chloride and the first incubation temperature is from 18° C. to 30° C. or from 20° C. to 25° C.
Embodiment 8 is the method of any one of the preceding embodiments, wherein the acid used in the first incubation stage is sulfuric acid and the first incubation temperature is from 2° C. to 21° C., 2° C. to 18° C., 4° C. to 21° C., 4° cto 18° C., or 4° C. to 10° C.
Embodiment 9 is the method of any one of the preceding embodiments, wherein no external heat is applied to the first suspension.
Embodiment 10 is the method of any one of the preceding embodiments, wherein the sample is not subjected to hydrolysis at temperatures above 90° C.
Embodiment 11 is the method of any one of the preceding embodiments, wherein the supernatant is substantially free of furfural and hydroxymethylfurfural.
Embodiment 12 is the method of any one of the preceding embodiments, wherein the method does not include enzymatic hydrolysis.
Embodiment 13 is the method of any one of the preceding embodiments, wherein the method causes hydrolysis of the sample, and wherein hydrolysis consists of the first incubation stage and the second incubation stage.
Embodiment 14 is the method of any one of the preceding embodiments, wherein the method comprises calculating hemicellulose as:

$$\% dw Total Hemicellulose = ( \frac{( \frac{X - Y + S - T}{100} ) * ( A + R )}{A} ) / ( 1.136 * ( \frac{D}{100} ) ) * 100 ,$$

where:
R is a total weight of liquid added to the sample;
A is mass of the sample;
D is wt-% total solids content of the sample;
S is the arabinose content of the sample in wt-%;
T is an arabinose content of a blank in wt-%;
X is a combined xylose, galactose, and mannose content of the sample in wt-%; and
Y is a combined xylose, galactose, and mannose content of the blank in wt-%.
Embodiment 15 is the method of any one of the preceding embodiments, wherein the method comprises calculating cellulose as:

$$\% dw Cellulose = \% dw Cellulosic Content - \% dw Hemicellulose$$

Embodiment 16 is the method of any one of the preceding embodiments comprising determining an amount of ethanol produced from cellulosic content as % D3 RIN.
Embodiment 17 is a method for quantifying a cellulosic component in a sample, the method comprising: incubating the sample at a first incubation stage at a first acid concentration and first temperature, followed by a second incubation stage at a second acid concentration that is lower than the first acid concentration and at a second temperature that is higher than the first temperature and below 98° C.; separating a supernatant from the incubated sample; analyzing the supernatant for total content of xylose, mannose, galactose, and arabinose; and calculating, based on the analysis, the amount of the cellulosic component, wherein the cellulosic component comprises hemicellulose, cellulose, or both.
Embodiment 18 is the method of embodiment 17, wherein the sample is corn-based or sorghum-based.
Embodiment 19 is the method of any one of the preceding embodiments, wherein the sample comprises raw ground corn.
Embodiment 20 is the method of any one of the preceding embodiments, wherein the sample comprises a starting material, an intermediate product, or a final product from a corn-to-ethanol production process.
Embodiment 21 is the method of any one of the preceding embodiments, wherein the amount of sample is mixed with an amount of the first aqueous acid that is from 8 to 12 parts of aqueous acid to every 1 part sample.
Embodiment 22 is the method of any one of the preceding embodiments, wherein the sample is not subjected to hydrolysis at temperatures above 90° C.
Embodiment 23 is the method of any one of the preceding embodiments, wherein the first acid concentration is greater than 40 wt-% wt-% and the second acid concentration is less than 40 wt-%.
Embodiment 24 is the method of any one of the preceding embodiments, wherein the supernatant is substantially free of furfural and hydroxymethylfurfural.
Embodiment 25 is the method of any one of the preceding embodiments, wherein the method does not include enzymatic hydrolysis.
Embodiment 26 is the method of any one of the preceding embodiments, wherein the method causes hydrolysis of the sample, and wherein hydrolysis consists of the incubating of the suspension at the incubation temperature for the incubation period.
Embodiment 27 is the method of any one of the preceding embodiments, wherein the method comprises calculating hemicellulose as:

$$\% dw Total Hemicellulose = ( \frac{( \frac{X - Y + S - T}{100} ) * ( A + R )}{A} ) / ( 1.136 * ( \frac{D}{100} ) ) * 100 ,$$

where:
R is a total weight of liquid added to the sample;
A is mass of the sample,
D is wt-% total solids content of the sample;
S is the arabinose content of the sample in wt-%;
T is an arabinose content of a blank in wt-%;
X is a combined xylose, galactose, and mannose content of the sample in wt-%; and
Y is a combined xylose, galactose, and mannose content of the blank in wt-%.
Embodiment 28 is the method of any one of the preceding embodiments, wherein the method comprises calculating cellulose as: % dw Cellulose=% dw Cellulosic content−% dw Hemicellulose
Embodiment 29 is the method of any one of the preceding embodiments comprising determining an amount of ethanol produced from cellulosic content as % D3 RIN.
Embodiment 30 is an analysis result comprising a hemicellulose concentration of a sample obtained by:
preparing a first suspension by mixing an amount of sample with aqueous acid comprising zinc chloride at a concentration of 60 wt-% to 80 wt-% or sulfuric acid at a concentration of 40 wt-% to 75 wt-%, based on a total weight of the first suspension;
incubating the first suspension in a first incubation stage at a first incubation temperature of less than 40° C. for 10 min to 60 min;
adding water or aqueous sulfuric acid to the incubated first suspension to form a second suspension having a concentration of 5 wt-% to 40 wt-% of sulfuric acid based on a total weight of the second suspension;
incubating the second suspension in a second incubation stage at a second incubation temperature of 50° C. or greater for an incubation period of 120 min to 500 min,
wherein the sample is not subjected to hydrolysis at temperatures above 98° C.;
separating a supernatant from the incubated second suspension; analyzing the supernatant for total content of xylose, mannose, galactose, and arabinose; and
calculating, based on the analysis, the amount of the hemicellulose in the sample.
Embodiment 31 is the analysis result of embodiment 30, wherein the first incubation temperature is 35° C. or less, 30° C. or less, or 25° C. or less, or wherein the first incubation temperature is from 2° C. to 21° C., 2° C. to 18° C., 4° C. to 21° C., 4° C. to 18° C., or 4° C. to 10° C.
Embodiment 32 is the analysis result of embodiment 30 or 31, wherein the acid used in the first incubation stage is zinc chloride and the first incubation temperature is from 18° C. to 30° C. or from 20° C. to 25° C.
Embodiment 33 is the method of embodiment 30 or 31, wherein the acid used in the first incubation stage is sulfuric acid and the first incubation temperature is from 2° C. to 21° C., 2° C. to 18° C., 4° C. to 21° C., 4° C. to 18° C., or 4° C. to 10° C.
Embodiment 34 is an analysis result comprising a hemicellulose concentration of a sample, the hemicellulose concentration obtained by a method including hydrolysis of the sample, the hydrolysis consisting of the first incubation stage and the second incubation stage.
Embodiment 35 is a method for quantifying hemicellulose in a sample of cellulosic material, the method comprising:
preparing a first suspension by mixing an amount of sample with first aqueous acid comprising zinc chloride at a concentration of 60 wt-% to 80 wt-% based on a total weight of the first suspension;
incubating the first suspension in a first incubation stage at a first incubation temperature of less than 40° C. for 10 min to 60 min;
adding aqueous sulfuric acid to the incubated first suspension to form a second suspension having a concentration of 5 wt-% to 40 wt-% of sulfuric acid based on a total weight of the second suspension;
incubating the second suspension in a second incubation stage at a second incubation temperature of 50° C. or greater for an incubation period of 120 min to 500 min,
wherein the sample is not subjected to hydrolysis at temperatures above 98° C.;
separating a supernatant from the incubated second suspension;
analyzing the supernatant for total content of xylose, mannose, galactose, and arabinose; and
calculating, based on the analysis, the amount of the hemicellulose.
Embodiment 36 is the method of embodiment 35, wherein the sample is corn-based or sorghum-based.
Embodiment 37 is the method of embodiment 35 or 36, wherein the sample comprises raw ground corn.
Embodiment 38 is the method of any one of embodiments 35 to 37, wherein the sample comprises a starting material, an intermediate product, or a final product from a corn-to-ethanol production process.
Embodiment 39 is the method of any one of embodiments 35 to 3, wherein the amount of sample is mixed with an amount of the first aqueous acid that is from 8 to 12 parts of aqueous acid to every 1 part sample.
Embodiment 40 is the method of any one of embodiments 35 to 39, wherein the first incubation temperature is 35° C. or less, 30° C. or less, or 25° C. or less, or ambient temperature, or ranges from 18° C. to 30° C. or from 20° C. to 25° C.
Embodiment 41 is the method of any one of embodiments 35 to 40, wherein no external heat is applied to the first suspension.
Embodiment 42 is the method of any one of embodiments 35 to 41, wherein the sample is not subjected to hydrolysis at temperatures above 90° C.
Embodiment 43 is the method of any one of embodiments 35 to 42, wherein the supernatant is substantially free of furfural and hydroxymethylfurfural.
Embodiment 44 is the method of any one of embodiments 35 to 43, wherein the method does not include enzymatic hydrolysis.
Embodiment 45 is the method of any one of embodiments 35 to 44, wherein the method causes hydrolysis of the sample, and wherein hydrolysis consists of the first incubation stage and the second incubation stage.
Embodiment 46 is the method of any one of embodiments 35 to 45, wherein the method comprises calculating hemicellulose as:

$$\% dw Total Hemicellulose = ( \frac{( \frac{X - Y + S - T}{100} ) * ( A + R )}{A} ) / ( 1.136 * ( \frac{D}{100} ) ) * 100 ,$$

where:
R is a total weight of liquid added to the sample;
A is mass of the sample;
D is wt-% total solids content of the sample;
S is the arabinose content of the sample in wt-%;
T is an arabinose content of a blank in wt-%;
X is a combined xylose, galactose, and mannose content of the sample in wt-%; and
Y is a combined xylose, galactose, and mannose content of the blank in wt-%.
Embodiment 47 is the method of any one of embodiments 35 to 46, wherein the method comprises calculating cellulose as:

$$\% dw Cellulose = \% dw Cellulosic Content - \% dw Hemicellulose$$

Embodiment 48 is the method of any one of embodiments 35 to 47 comprising determining an amount of ethanol produced from cellulosic content as % D3 RIN.
Embodiment 49 is an analysis result comprising a hemicellulose concentration of a sample obtained by:
preparing a first suspension by mixing an amount of sample with aqueous acid comprising zinc chloride at a concentration of 60 wt-% to 80 wt-% based on a total weight of the first suspension;
incubating the first suspension in a first incubation stage at a first incubation temperature of less than 40° C. for 10 min to 60 min;
adding aqueous sulfuric acid to the incubated first suspension to form a second suspension having a concentration of 5 wt-% to 40 wt-% of sulfuric acid based on a total weight of the second suspension;
incubating the second suspension in a second incubation stage at a second incubation temperature of 50° C. or greater for an incubation period of 120 min to 500 min,
wherein the sample is not subjected to hydrolysis at temperatures above 98° C.;
separating a supernatant from the incubated second suspension; analyzing the supernatant for total content of xylose, mannose, galactose, and arabinose; and calculating, based on the analysis, the amount of the hemicellulose in the sample.
Embodiment 50 is the analysis result of embodiment 49, wherein the first incubation temperature is 35° C. or less, 30° C. or less, or 25° C. or less, or ambient temperature, or ranges from 18° C. to 30° C. or from 20° C. to 25° C.
Embodiment 51 is a method for quantifying hemicellulose in a sample of cellulosic material, the method comprising:
preparing a first suspension by mixing an amount of sample with first aqueous acid comprising sulfuric acid at a concentration of 40 wt-% to 75 wt-%, based on a total weight of the first suspension;
incubating the first suspension in a first incubation stage at a first incubation temperature of less than 40° C. for 10 min to 60 min;
adding water or aqueous sulfuric acid to the incubated first suspension to form a second suspension having a concentration of 5 wt-% to 40 wt-% of sulfuric acid based on a total weight of the second suspension;
incubating the second suspension in a second incubation stage at a second incubation temperature of 50° C. or greater for an incubation period of 120 min to 500 min,
wherein the sample is not subjected to hydrolysis at temperatures above 98° C.;
separating a supernatant from the incubated second suspension:
analyzing the supernatant for total content of xylose, mannose, galactose, and arabinose; and
calculating, based on the analysis, the amount of the hemicellulose
Embodiment 52 is the method of embodiment 51, wherein the sample is corn-based or sorghum-based.
Embodiment 53 is the method of embodiment 51 or 52, wherein the sample comprises raw ground corn
Embodiment 54 is the method of any one of embodiments 51 to 53, wherein the sample comprises a starting material, an intermediate product, or a final product from a corn-to-ethanol production process.
Embodiment 55 is the method of any one of embodiments 51 to 54, wherein the amount of sample is mixed with an amount of the first aqueous acid that is from 8 to 12 parts of aqueous acid to every 1 part sample.
Embodiment 56 is the method of any one of embodiments 51 to 55, wherein the first incubation temperature is 35° C. or less, 30° C. or less, or 25° C. or less, or wherein the first incubation temperature is from 2° C. to 21° C., 2° C. to 18° C., 4° C. to 21° C., 4° C. to 18° C., or 4° C. to 10° C.
Embodiment 57 is the method of any one of embodiments 51 to 56, wherein no external heat is applied to the first suspension.
Embodiment 58 is the method of any one of embodiments 51 to 57, wherein the sample is not subjected to hydrolysis at temperatures above 90° C.
Embodiment 59 is the method of any one of embodiments 51 to 58, wherein the supernatant is substantially free of furfural and hydroxymethylfurfural.
Embodiment 60 is the method of any one of embodiments 51 to 59, wherein the method does not include enzymatic hydrolysis.
Embodiment 61 is the method of any one of embodiments 51 to 60, wherein the method causes hydrolysis of the sample, and wherein hydrolysis consists of the first incubation stage and the second incubation stage.
Embodiment 62 is the method of any one of embodiments 51 to 61, wherein the method comprises calculating hemicellulose as:

$$\% dw Total Hemicellulose = ( \frac{( \frac{X - Y + S - T}{100} ) * ( A + R )}{A} ) / ( 1.136 * ( \frac{D}{100} ) ) * 100 ,$$

where:
R is a total weight of liquid added to the sample;
A is mass of the sample,
D is wt-% total solids content of the sample;
S is the arabinose content of the sample in wt-%;
T is an arabinose content of a blank in wt-%;
X is a combined xylose, galactose, and mannose content of the sample in wt-%; and
Y is a combined xylose, galactose, and mannose content of the blank in wt-%.
Embodiment 63 is the method of any one of embodiments 51 to 62, wherein the method comprises calculating cellulose as:

% dw Cellulose=% dw Cellulosic content−% dw Hemicellulose
Embodiment 64 is the method of any one of embodiments 51 to 63 comprising determining an amount of ethanol produced from cellulosic content as % D3 RIN.
Embodiment 65 is an analysis result comprising a hemicellulose concentration of a sample obtained by:
preparing a first suspension by mixing an amount of sample with aqueous acid comprising sulfuric acid at a concentration of 40 wt-% to 75 wt-%, based on a total weight of the first suspension;
incubating the first suspension in a first incubation stage at a first incubation temperature of less than 40° C. for 10 min to 60 min;
adding water or aqueous sulfuric acid to the incubated first suspension to form a second suspension having a concentration of 5 wt-% to 40 wt-% of sulfuric acid based on a total weight of the second suspension;
incubating the second suspension in a second incubation stage at a second incubation temperature of 50° C. or greater for an incubation period of 120 min to 500 min,
wherein the sample is not subjected to hydrolysis at temperatures above 98° C.:
separating a supernatant from the incubated second suspension; analyzing the supernatant for total content of xylose, mannose, galactose, and arabinose; and
calculating, based on the analysis, the amount of the hemicellulose in the sample.
Embodiment 66 is the analysis result of embodiment 65, wherein the first incubation temperature is 35° C. or less, 30° C. or less, or 25° C. or less, or wherein the first incubation temperature is from 2° C. to 21° C., 2° C. to 18° C., 4° C. to 21° C., 4° C. to 18° C., or 4° C. to 10° C.

### EXPERIMENTAL

Various samples were tested using conventional analytical methods known in the art and the analytical methods described here.

### Example 1

Various corn-based before conversion (“BC”) and after conversion (“AC”) samples were tested using the method of the present disclosure, and the results were compared to results achieved by conventional commercially available methods.

The amount of hemicellulose in the sample was calculated using formula (I). The amount of cellulose in the sample was calculated using formula (II)A. The cellulosic content was obtained by conventional commercially available methods.

The converted % D3 RINs were calculated from the commercially available results for cellulosic content. Additionally, the % D3 RINs were separately calculated for those derived from the hemicellulose fraction by using the hemicellulose results from the method of the present disclosure and the cellulose result as calculated using commercially available cellulosic content and the hemicellulose by the method of the present disclosure. The results are shown in TABLES 1A-1C.

The results in TABLE 1A are given as % dry weight.

TABLE 1A
Commercially available method results.

| Sample ID | AOAC Ash | Starch | Cellulosic Content |
|---|---|---|---|
| 1BC | 2.0 | 70.3 | 11.9 |
| 1AC | 6.1 | 4.6 | 29.6 |
| 2BC | 1.9 | 70.9 | 11.4 |
| 2AC | 6.0 | 4.7 | 29.4 |
| 3BC | 1.9 | 70.6 | 11.5 |
| 3AC | 6.0 | 4.6 | 29.4 |
| 4BC | 2.3 | 68.7 | 12.4 |
| 4AC | 7.4 | 5.8 | 31.3 |
| 5BC | 2.3 | 69.0 | 12.4 |
| 5AC | 7.4 | 5.3 | 31.4 |
| 6BC | 2.0 | 69.4 | 12.1 |
| 6AC | 6.1 | 7.5 | 31.7 |
| 7BC | 2.0 | 69.6 | 11.9 |
| 7AC | 6.1 | 7.3 | 31.4 |
| 8BC | 2.0 | 69.6 | 11.7 |
| 8AC | 6.1 | 7.4 | 31.4 |

In TABLE 1B, cellulose is calculated by subtracting the hemicellulose from the total cellulosic content. The results in TABLE 1B are given as % dry weight.

TABLE 1B
Present disclosure method results.

| | | Cellulose |
|---|---|---|
| | | (Cellulosic Content − |
| Sample ID | Hemicellulose | Hemicellulose) |
| 1BC | 4.8 | 7.0 |
| 1AC | 13.9 | 15.7 |
| 2BC | 4.8 | 6.5 |
| 2AC | 13.9 | 15.5 |
| 3BC | 4.9 | 6.7 |
| 3AC | 13.9 | 15.5 |
| 4BC | 4.9 | 7.5 |
| 4AC | 14.2 | 17.0 |
| 5BC | 5.0 | 7.3 |
| 5AC | 14.7 | 16.8 |
| 6BC | 4.8 | 7.2 |
| 6AC | 13.9 | 17.8 |
| 7BC | 4.8 | 7.1 |
| 7AC | 13.7 | 17.7 |
| 8BC | 4.8 | 6.9 |
| 8AC | 13.7 | 17.7 |

In TABLE 1C, the % of gallons of ethanol derived from the cellulosic content and cellulosic components (“% D3 RINs”) are shown. The % D3 RINs are calculated from the commercially available cellulosic content, hemicellulose, and cellulose results above, using EPA prescribed calculations.

TABLE 1C
% D3 RINs results.

| | % D3 RINs | | % D3 RINs from |
|---|---|---|---|
| | from | % D3 RINs | Cellulose |
| Sample | Cellulosic | from | (Cellulosic Content − |
| ID | Content | Hemicellulose | Hemicellulose) |
| 1BC | 3.23% | 0.47% | 2.76% |
| 2BC | 2.68% | 0.55% | 2.14% |
| 3BC | 2.86% | 0.52% | 2.34% |
| 4BC | 3.76% | 0.69% | 3.07% |
| 5BC | 3.56% | 0.65% | 2.90% |
| 6BC | 2.76% | 0.60% | 2.16% |
| 7BC | 2.64% | 0.59% | 2.05% |
| 8BC | 2.33% | 0.51% | 1.81% |

By using the method of the present disclosure, it is now possible to speciate the gallons of cellulosic ethanol (% D3 RINs) derived from hemicellulose and cellulose. This is of great utility when trying to determine the efficacy of process changes, cellulase and hemicellulase enzyme treatments, and the efficacy of the various microorganisms used in fermentation to convert both pentose and hexose monosaccharides to ethanol.

### Example 2

A mixture of pentose and hexose monosaccharides was prepared. The mixture contained the following pentose monosaccharides: xylose and arabinose, and the following hexose monosaccharides: glucose, mannose, and galactose. Xylose, arabinose, mannose, and galactose are monosaccharides derived from the hemicellulose fraction of corn kernel fiber. Glucose is a monosaccharide derived from both the starch and cellulose fractions in corn and corn fiber.

Each of the five monosaccharides was blended at a concentration of 2 wt-% into an aqueous fermentation media prepared by blending 0.5 wt-% yeast extract solution and pH adjusting to 5.0±0.2. Saccharomyces cerevisiae yeast was added at a concentration of 0.02 grams per 100 grams of fermentation media. Urea was added at a concentration of 0.1 ml of a 40 wt-% urea solution per 100 grams of fermentation media. An antimicrobial (Lactocide 247) was added at concentration of 0.006 grams per 100 grams of fermentation media.

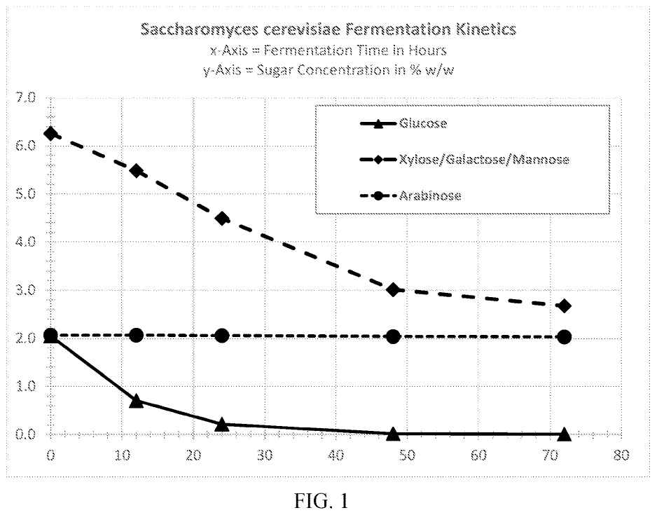

All fermentations were incubated at 32° C. for 72 hours. Samples were taken from all fermentations at 0, 24, 48, and 72 hours of fermentation. From each sample, a supernatant was separated from the suspension by centrifuging and filtering using a 25 μm filter. The supernatant was analyzed for glucose, xylose, mannose, galactose, and arabinose content using HPLC with an AMINEX HPX-87H column available from Bio-Rad Laboratories, Inc. in Hercules, CA. The results are presented in FIG. 1.

It was observed that the Saccharomyces cerevisiae yeast utilized all the glucose over the course of the 72-hour fermentation. It was also observed that the Saccharomyces cerevisiae yeast utilized nearly all of the other hexose monosaccharides (mannose and galactose) as well over the course of the 72-hour fermentation. It was also observed that the Saccharomyces cerevisiae yeast did not utilize any of the pentose sugars (xylose and arabinose) over the course of the 72-hour fermentation—this is consistent with what the scientific community would expect from a non-genetically modified variant of Saccharomyces cerevisiae.

These findings are significant in the context of the method of the present disclosure because of the proof that Saccharomyces cerevisiae does utilize other hexose monosaccharides that can be contributed by the hemicellulose, e.g., in #2 Yellow corn. Given this, the amount of cellulosic ethanol that can be derived from a corn ethanol plant that is using non-genetically modified variant of Saccharomyces cerevisiae could be increased by the degree of galactose and mannose present in the corn fiber.

By using the method of present disclosure, it is now possible to speciate the gallons of cellulosic ethanol (% D3 RINs) derived from hemicellulose and cellulose. This is of great utility when trying to determine the efficacy of cellulase and hemicellulase enzyme treatments and the efficacy of the various microorganisms used in fermentation to convert both pentose and hexose monosaccharides to ethanol.

### Example 3

Various samples of #2 Yellow Corn were tested using the method of the present disclosure, and the results were compared to results achieved by conventional commercially available methods.

The amount of hemicellulose in the sample was calculated using formula (I). The amount of cellulose in the sample was calculated using formula (II)A. The results in TABLE 2 are given as % dry weight.

TABLE 2

| | | | | | Cellulose - Hemicellulose | |
|---|---|---|---|---|---|---|
| | Commercially Available | | | | Ratio | |
| | Method Results | | Method of Present | | Percent of | Percent of |
| | | Cellulosic | Disclosure Results | | Fiber that is | Fiber that is |
| | Starch | Content | Hemicell. | Cellulose | Cellulose | Hemicellulose |
| Sample ID | % dw | % dw | % dw | % dw | % | % |
| Sample #1 | 76.64 | 9.91 | 5.2 | 4.7 | 48% | 52% |
| Sample #2 | 75.96 | 11.33 | 5.0 | 6.3 | 56% | 44% |
| Sample #3 | 76.2 | 10.8 | 5.0 | 5.8 | 54% | 46% |
| Sample #4 | 76.3 | 10.9 | 4.8 | 6.1 | 56% | 44% |
| Sample #5 | 75.6 | 11.5 | 5.0 | 6.5 | 56% | 44% |
| Sample #6 | 76.8 | 10.2 | 4.9 | 5.3 | 52% | 48% |
| Sample #7 | 76.4 | 10.7 | 4.9 | 5.8 | 54% | 46% |
| Sample #8 | 75.8 | 11.4 | 5.1 | 6.3 | 55% | 45% |
| Sample #9 | 76.0 | 11.0 | 5.0 | 6.0 | 55% | 45% |
| Sample #10 | 76.6 | 10.2 | 4.8 | 5.3 | 52% | 48% |
| Sample #11 | 75.8 | 10.8 | 4.9 | 6.0 | 55% | 45% |
| Sample #12 | 75.4 | 11.5 | 5.0 | 6.5 | 57% | 43% |
| Sample #13 | 75.7 | 11.4 | 5.0 | 6.4 | 56% | 44% |
| Sample #14 | 76.0 | 10.5 | 5.0 | 5.5 | 52% | 48% |

TABLE 3

| | Theoretical Yield Potential | | | | | | | | |
|---|---|---|---|---|---|---|---|---|---|
| | gal/bushel | | | | | | % D3 RINs | | |
| | From | From | From | From Total | From Total | From Total | From | From | From Total |
| Sample ID | Starch | Cellulose | Hemicell. | Kernel Fiber | Glucan | Carbs | Cellulose | Hemicell. | Kernel Fiber |
| Sample #1 | 2.83 | 0.17 | 0.19 | 0.37 | 3.01 | 3.20 | 5.4% | 6.0% | 11.5% |
| Sample #2 | 2.81 | 0.23 | 0.18 | 0.42 | 3.04 | 3.23 | 7.3% | 5.7% | 13.0% |
| Sample #3 | 2.82 | 0.21 | 0.19 | 0.40 | 3.03 | 3.21 | 6.6% | 5.8% | 12.4% |
| Sample #4 | 2.82 | 0.22 | 0.18 | 0.40 | 3.04 | 3.22 | 7.0% | 5.5% | 12.5% |
| Sample #5 | 2.79 | 0.24 | 0.19 | 0.43 | 3.03 | 3.22 | 7.4% | 5.8% | 13.2% |
| Sample #6 | 2.84 | 0.20 | 0.18 | 0.38 | 3.03 | 3.22 | 6.1% | 5.7% | 11.8% |
| Sample #7 | 2.82 | 0.21 | 0.18 | 0.39 | 3.03 | 3.22 | 6.6% | 5.6% | 12.2% |
| Sample #8 | 2.80 | 0.23 | 0.19 | 0.42 | 3.03 | 3.22 | 7.2% | 5.9% | 13.1% |
| Sample #9 | 2.81 | 0.22 | 0.18 | 0.40 | 3.03 | 3.21 | 6.9% | 5.7% | 12.6% |
| Sample #10 | 2.83 | 0.20 | 0.18 | 0.38 | 3.03 | 3.21 | 6.1% | 5.6% | 11.7% |
| Sample #11 | 2.80 | 0.22 | 0.18 | 0.40 | 3.02 | 3.20 | 6.9% | 5.6% | 12.5% |
| Sample #12 | 2.79 | 0.24 | 0.18 | 0.42 | 3.03 | 3.21 | 7.5% | 5.7% | 13.2% |
| Sample #13 | 2.80 | 0.24 | 0.18 | 0.42 | 3.03 | 3.22 | 7.4% | 5.7% | 13.1% |
| Sample #14 | 2.81 | 0.20 | 0.19 | 0.39 | 3.01 | 3.19 | 6.3% | 5.8% | 12.1% |

By using the method of the present disclosure, it is now possible to determine the hemicellulose and cellulose ratio for samples of grain, intermediate process streams, and final feed products from ethanol production facilities and animal feed manufacturers. Additionally, by using the method of present disclosure, it is now possible to determine the theoretical yield potential of all the carbohydrate sources separately for any given grain feedstock—starch, cellulose, and hemicellulose. This is of great utility when trying to determine the efficacy of process changes, cellulase and hemicellulase enzyme treatments, and the efficacy of the various microorganisms used in fermentation to convert both pentose and hexose monosaccharides to ethanol. Additionally, this speciated fiber information is useful to nutritionist when formulating feed rations.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A method for quantifying hemicellulose in a sample of cellulosic material, the method comprising:
preparing a first suspension by mixing an amount of sample with first aqueous acid comprising zinc chloride at a concentration of 60 wt-% to 80 wt-% or sulfuric acid at a concentration of 40 wt-% to 75 wt-%, based on a total weight of the first suspension;
incubating the first suspension in a first incubation stage at a first incubation temperature of less than 40° C. for 10 min to 60 min;
adding water or aqueous sulfuric acid to the incubated first suspension to form a second suspension having a concentration of 5 wt-% to 40 wt-% of sulfuric acid based on a total weight of the second suspension;
incubating the second suspension in a second incubation stage at a second incubation temperature of 50° C. or greater for an incubation period of 120 min to 500 min,
wherein the sample is not subjected to hydrolysis at temperatures above 98° C.;
separating a supernatant from the incubated second suspension;
analyzing the supernatant for total content of xylose, mannose, galactose, and arabinose; and
calculating, based on the analysis, the amount of the hemicellulose.

2. The method of claim 1, wherein the sample is corn-based.

3. The method of claim 2, wherein the sample comprises raw ground corn.

4. (canceled)

5. The method of claim 1, wherein the amount of sample is mixed with an amount of the first aqueous acid that is from 8 to 12 parts of aqueous acid to every 1 part sample.

6. (canceled)

7. The method of claim 1, wherein no external heat is applied to the first suspension.

8. The method of claim 1, wherein the sample is not subjected to hydrolysis at temperatures above 90° C.

9. The method of claim 1, wherein the supernatant is substantially free of furfural and hydroxymethylfurfural.

10. The method of claim 1, wherein the method does not include enzymatic hydrolysis.

11. The method of claim 1, wherein the method causes hydrolysis of the sample, and wherein hydrolysis consists of the first incubation stage and the second incubation stage.

12. The method of claim 1, wherein the method comprises calculating hemicellulose as:
$$\% dw Total Hemicellulose = ( \frac{( \frac{X - Y + S - T}{100} ) * ( A + R )}{A} ) / ( 1.136 * ( \frac{D}{100} ) ) * 100 ,$$
where:
R is a total weight of liquid added to the sample;
A is mass of the sample;
D is wt-% total solids content of the sample;
S is the arabinose content of the supernatant in wt-%;
T is an arabinose content of a blank in wt-%;
X is a combined xylose, galactose, and mannose content of the supernatant in wt-%; and
Y is a combined xylose, galactose, and mannose content of the blank in wt-%.

13. (canceled)

14. (canceled)

15. A method for quantifying a cellulosic component in a sample, the method comprising:
incubating the sample at a first incubation stage at a first acid concentration and first temperature, followed by a second incubation stage at a second acid concentration that is lower than the first acid concentration and at a second temperature that is higher than the first temperature and below 98° C.;
separating a supernatant from the incubated sample;
analyzing the supernatant for total content of xylose, mannose, galactose, and arabinose; and
calculating, based on the analysis, the amount of the cellulosic component, wherein the cellulosic component comprises hemicellulose, cellulose, or both.

16. (canceled)

17. The method of claim 15, wherein the sample comprises raw ground corn.

18. (canceled)

19. The method of claim 15, wherein the amount of sample is mixed with an amount of the first aqueous acid that is from 8 to 12 parts of aqueous acid to every 1 part sample.

20. The method of claim 15, wherein the sample is not subjected to hydrolysis at temperatures above 90° C.

21. The method of claim 15, wherein the first acid concentration is greater than 40 wt-% wt-% and the second acid concentration is less than 40 wt-%.

22. The method of claim 15, wherein the supernatant is substantially free of furfural and hydroxymethylfurfural.

23. The method of claim 15, wherein the method does not include enzymatic hydrolysis.

24. The method of claim 15, wherein the method causes hydrolysis of the sample, and wherein hydrolysis consists of the incubating of the suspension at the incubation temperature for the incubation period.

25. The method of claim 15, wherein the method comprises calculating hemicellulose as:
$$\% dw Total Hemicellulose = ( \frac{( \frac{X - Y + S - T}{100} ) * ( A + R )}{A} ) / ( 1.136 * ( \frac{D}{100} ) ) * 100 ,$$
where:
R is a total weight of liquid added to the sample;
A is mass of the sample;
D is wt-% total solids content of the sample;
S is the arabinose content of the supernatant in wt-%;
T is an arabinose content of a blank in wt-%;
X is a combined xylose, galactose, and mannose content of the supernatant in wt-%; and
Y is a combined xylose, galactose, and mannose content of the blank in wt-%.

26. (canceled)

27. (canceled)

28. An analysis result comprising a hemicellulose concentration of a sample obtained by:
preparing a first suspension by mixing an amount of sample with aqueous acid comprising zinc chloride at a concentration of 60 wt-% to 80 wt-% or sulfuric acid at a concentration of 40 wt-% to 75 wt-%, based on a total weight of the first suspension;
incubating the first suspension in a first incubation stage at a first incubation temperature of less than 40° C. for 10 min to 60 min;
adding water or aqueous sulfuric acid to the incubated first suspension to form a second suspension having a concentration of 5 wt-% to 40 wt-% of sulfuric acid based on a total weight of the second suspension;
incubating the second suspension in a second incubation stage at a second incubation temperature of 50° C. or greater for an incubation period of 120 min to 500 min,
wherein the sample is not subjected to hydrolysis at temperatures above 98° C.;
separating a supernatant from the incubated second suspension; analyzing the supernatant for total content of xylose, mannose, galactose, and arabinose; and
calculating, based on the analysis, the amount of the hemicellulose in the sample.

29. (canceled)
